(19) United States
(12) Patent Application Publication
(10) Pub. No.: US 2023/0210681 A1
(43) Pub. Date: Jul. 6, 2023

(54) SLING DEVICE FOR SUPPORTING THE LEG AND FOOT OF A USER AND METHODS OF MAKING AND USING THE SAME

(71) Applicant: Rachel M. Lance, Durham, NC (US)

(72) Inventor: Rachel M. Lance, Durham, NC (US)

(21) Appl. No.: 18/093,616

(22) Filed: Jan. 5, 2023

Related U.S. Application Data
(60) Provisional application No. 63/296,921, filed on Jan. 6, 2022.

(51) Int. Cl. A61F 5/37

(52) U.S. Cl. CPC A61F 5/3715

(57) ABSTRACT

The invention is directed to a sling that can be used to support the foot and leg of a user off the ground, in a forward position in front of the user. Advantageously, the sling can be used as a mobility aid for users with a limited ability to bend their knee or those that need to use crutches, canes, walkers, and the like. The sling device includes a foot support loop sized and shaped to accommodate a user's foot. The support loop is operatively attached to an adjustable strap that wraps around the user's shoulder and/or back for support. Advantageously, when a user's foot is positioned within the loop and the strap is configured around the user's shoulder and/or back, the user's foot and leg are lifted in a forward direction, ensuring that the foot is positioned off of the ground.

### CROSS-REFERENCE TO RELATED APPLICATIONS

This application claims priority to U.S. Provisional Patent Application No. 63/296,921, filed Jan. 6, 2022, the entire content of which is incorporated by reference herein.

### TECHNICAL FIELD

The presently disclosed subject matter is generally directed to a sling device that can be used to support the leg and foot of a user. The presently disclosed subject matter is also directed to methods of making and using the sling device.

### BACKGROUND

Restraining sling devices have been commonly used for many years to restrict movement of a limb, such as an arm. Specifically, the sling acts as a restraint, preventing or limiting movement of the limb after surgery or injury to allow the limb to heal properly and prevent further pain or injury. However, prior art slings have been primarily limited to use with injured arms. After surgery or other injury to the leg, a locking brace is often worn and crutches are used to move from one place to another. However, knee braces do not always work as intended, and lightweight or non-locking brace designs can slip or otherwise move from the correct position. Knee braces also tend to be bulky and add substantial weight to the user's leg. In addition, when using crutches with normal knee mobility, the tendency is to bend the knee so that the foot lifts backwards off the ground (flexion). However, when crutches are used with a locking knee brace, the knee is forced to remain straight (full extension) or provide only a limited, specific range of flexion. When flexion is limited in this manner, it becomes difficult to walk or use crutches, because the foot contacts the ground and often gets caught or caught on objects. To use crutches, these patients must manually lift their entire leg forward while the knee is locked in extension to raise the foot from the ground. Maintaining the forward lift of the leg while wearing a heavy knee or leg brace is physically taxing and results in severe strain to the muscles of the hip, limiting mobility of the patient. It would therefore be beneficial to provide a sling device for supporting the leg of a user while also keeping the leg raised in a forward position above the ground.

### SUMMARY

In some embodiments, the presently disclosed subject matter is directed to a sling for supporting the leg and foot of a user. Specifically, the sling comprises a loop with an interior opening, sized and shaped to house the foot (or a portion of the foot) of a user, whereby the loop is configured to transfer the weight of the user's leg to the sling. The sling also includes a strap defined by first and second ends, each end operatively attached to an end of the loop. In some embodiments, the strap may alternatively be sewn in a circle that passes through the end of the loop. The strap transfers weight from the loop to the patient's shoulder, back, or both. The strap length is less than the length between the strap and a floor, such that the loop is elevated from the floor in a forward direction relative to the rest of the user's body.

In some embodiments, the length of the strap is adjustable.

In some embodiments, the loop is constructed from a rigid or semi-rigid material.

In some embodiments, the strap includes one or more padded areas.

In some embodiments, an interior surface of the loop comprises a textured region.

In some embodiments, an interior surface of the loop comprises a padded lining.

In some embodiments, the loop central opening has a circular, oval, square, rectangular, or triangular shape.

In some embodiments, the loop has a length of about 4-12 inches.

In some embodiments, the loop is permanently or releasably attached to the strap.

In some embodiments, the strap has a length of about 20-60 inches.

In some embodiments, the strap is constructed from a flexible material.

In some embodiments, the presently disclosed subject matter is directed to a method of supporting a user's foot and leg. Specifically, the method comprises inserting a foot of user into the loop of the disclosed sling. Each end of the strap is attached to an end of the loop. The method includes securing the strap around the back, shoulder, or both of the user. The method includes adjusting the length of the strap such that the foot positioned within the loop is extended in front of the user and raised from the floor, whereby the user's foot is supported.

In some embodiments, a bottom surface of the user's foot comes into contact with an inner surface of the loop.

In some embodiments, the foot positioned within the loop is raised about 2-10 inches.

In some embodiments, the user's leg remains in a locked position.

In some embodiments, the method further comprises moving from one area to another with crutches.

In some embodiments, the presently disclosed subject matter is directed to a kit. Specifically, the kit includes a plurality of loops, each loop defined by an interior opening, sized and shaped to house the foot of a user. The kit includes a plurality of straps, each strap defined by first and second ends, each end operatively attached to an end of the loop. A user can select a specific type of loop and strap for forming into a leg sling, and the strap length is less than the length between the strap and a floor, such that the loop is elevated from the floor in a forward direction relative to the user.

### BRIEF DESCRIPTION OF THE DRAWINGS

FIG. 1 illustrates a support device in accordance with some embodiments of the presently disclosed subject matter.

FIG. 2a is a front plan view of a foot loop of a support device in accordance with some embodiments of the presently disclosed subject matter.

FIG. 2b is a side plan view of a foot configured in a foot loop in accordance with some embodiments of the presently disclosed subject matter.

FIG. 2c is a front plan view of a foot loop of a support device in accordance with some embodiments of the presently disclosed subject matter.

FIG. 2d is a front plan view of a foot loop of a support device in accordance with some embodiments of the presently disclosed subject matter.

FIG. 2e is a top plan view of a foot loop of a support device in accordance with some embodiments of the presently disclosed subject matter.

FIG. 3a is a fragmentary top plan view of a foot loop comprising textured regions in accordance with some embodiments of the presently disclosed subject matter.

FIG. 3b is a fragmentary top plan view of a foot loop comprising a lining in accordance with some embodiments of the presently disclosed subject matter.

FIGS. 4a-4e are cross-sectional views, illustrating foot loops of varying shapes in accordance with some embodiments of the presently disclosed subject matter.

FIG. 5a is a side plan view of a foot loop in accordance with some embodiments of the presently disclosed subject matter.

FIG. 5b is a front plan view of a foot loop in accordance with some embodiments of the presently disclosed subject matter.

FIG. 5c is a fragmentary side plan view of a foot loop in accordance with some embodiments of the presently disclosed subject matter.

FIG. 6a is a side plan view of a strap in accordance with some embodiments of the presently disclosed subject matter.

FIG. 6b is a perspective view of a strap attached to a loop in accordance with some embodiments of the presently disclosed subject matter.

FIG. 6c is a fragmentary front plan view of a strap attached to a loop with elastic therebetween in accordance with some embodiments of the presently disclosed subject matter.

FIG. 6d is a fragmentary front plan view of a padding attached to a strap in accordance with some embodiments of the presently disclosed subject matter.

FIG. 7 is a schematic illustrating a method of using the sling device in accordance with some embodiments of the presently disclosed subject matter.

FIG. 8a illustrates a sling device configured on a user in accordance with some embodiments of the presently disclosed subject matter.

FIG. 8b is a side view of a user's foot positioned in the sling device in accordance with some embodiments of the presently disclosed subject matter.

FIG. 8c is a front plan view of a user with a foot positioned in the sling device in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

As used herein, the term “and/or” includes any and all combinations of one or more of the associated listed items.

Relative terms such as “below” or “above” or “upper” or “lower” or “horizontal” or “vertical” may be used herein to describe a relationship of one element, layer, or region to another element, layer, or region as illustrated in the drawing figures. It will be understood that these terms and those discussed above are intended to encompass different orientations of the device in addition to the orientation depicted in the drawing figures.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

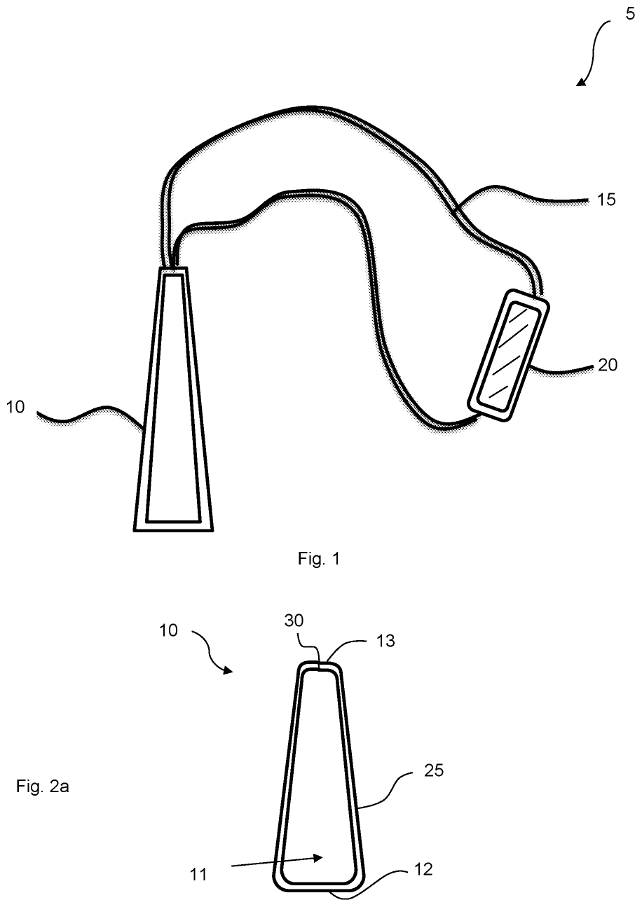

The presently disclosed subject matter is generally directed to a sling device for use with a user's foot and leg. Specifically, the disclosed sling can be used to support the foot and leg of a user off the ground, in a forward position in front of the user. Advantageously, the sling can be used with minimal effort from the user. Thus, the sling can be used as a mobility aid for users with a limited ability to bend their knee or those that need to use crutches, canes, walkers, and the like. FIG. 1 illustrates one embodiment of sling device 5 comprising foot support loop 10 sized and shaped to accommodate a user's foot. The support loop is operatively attached to adjustable strap 15 that wraps around the user's shoulder and/or back for support. In some embodiments, the strap can include one or more padded areas 20 that provide comfort to user when using the device. Advantageously, when a user's foot is positioned within the interior of loop 10 and the strap is configured around the user's shoulder and/or back, the user's foot and leg are lifted in a forward direction, ensuring that the foot is positioned off of the ground, as described in more detail below.

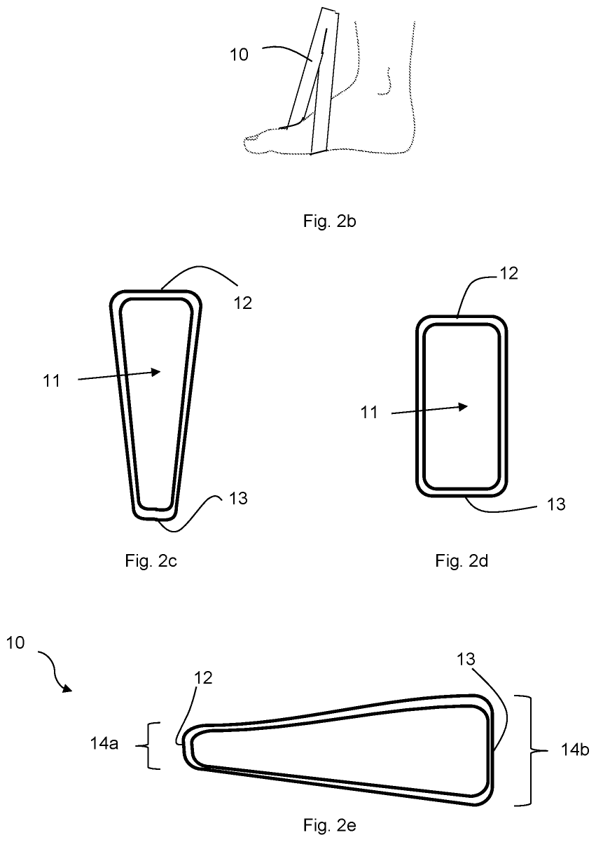

As set forth above, device 5 includes loop 10 configured to retain a user's foot. FIG. 2a illustrates one embodiment of loop 10 comprising a portion of material 25 joined at both ends 30 to form a closed structure with central opening 11. A user's foot is inserted into the central opening for support, as shown in FIG. 2b. In some embodiments, the loop can have a closed configuration with a single portion of material manufactured as an unbroken segment. Alternatively, the loop can have an open configuration with a portion of material that includes a break, such that the loop is not continuous. In some embodiments, the loop can include a wider end (e.g., bottom end 12) that accommodates a user's foot. The loop can also include a narrow end (e.g., top end 13) that facilitates joining the loop to material 25. However, the loop is not limited and can be configured such that the top end is wider compared to the bottom end as shown in FIG. 2c. Also, both the top and bottom end can have about the same width, as shown in FIG. 2d. When the width 14a of the top end differs from the bottom end width 14b, the difference can be about 5-700 percent less or more (e.g., at least/no more than about 5, 10, 15, 20, 25, 30, 35, 40, 45, 50, 100, 150, 200, 250, 300, 350, 400, 450, 500, 550, 600, 650, or 700 percent), as illustrated in FIG. 2e. Thus, if width 14a of the top end is about 1 inch, width 14b of the bottom end of the loop can be about 7 inches (e.g., about 700 percent of the width of the top end). In another example, when width 14a of the top end is about 4 inches, the width 14b of the bottom end can be about 3 inches (e.g., about 25 percent less).

Loop 10 can be constructed from any desired rigid or semi-rigid material. The term “rigid” refers to a material that is substantially or fully inflexible. The term “semi-rigid” refers to a material that is somewhat stiff or rigid but can be flexed or folded. Thus, the loop can be constructed from any of a wide variety of materials, such as (but not limited to) one or more of fabric, leather, plastic, rubber, silicone, rayon, polyester, and the like.

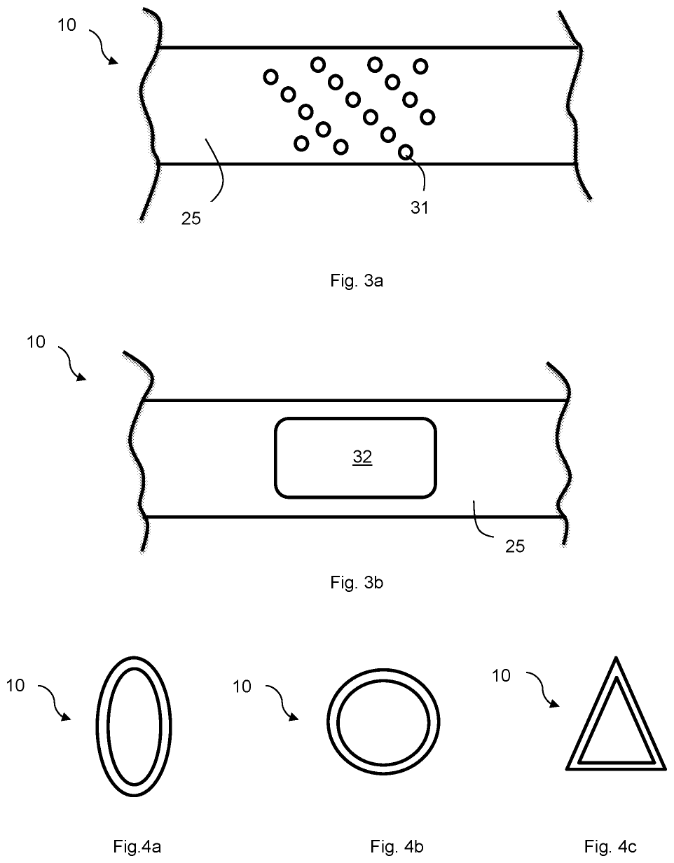

In some embodiments, all or a portion of the interior surface of the loop can include textured surface 31 to provide an anti-slip characteristic to the loop, as shown in FIG. 3a. In this way, the user's foot, sock, or shoe remains secured within the loop, and is unlikely to slip out. The term “textured surface” can include any area of rubber or silicon materials, raised regions, mesh, sticky or adhesive materials, and/or the like. In some embodiments, only the inner portion of the bottom end of the loop includes the textured surface (e.g., the region in contact with the user's foot). Thus, the textured surface can be configured only on the interior region of bottom end 13. The textured portion can comprise about 5-100 percent of the interior region of the loop (e.g., at least/no more than about 5, 10, 15, 20, 25, 30, 35, 40, 45, 50, 55, 60, 65, 70, 75, 80, 85, 90, 95, or 100 percent).

The interior portion of loop 10 in direct contact with the user's foot can also include lining 32 in all or a portion of the interior surface, as shown in FIG. 3b. The lining can provide an area of comfort to the user's foot as it rests within the interior of the loop. The lining can therefore include any soft or padded material, such as cotton, rubber, and the like. Thus, lining can be configured only on the interior region of bottom end 13.

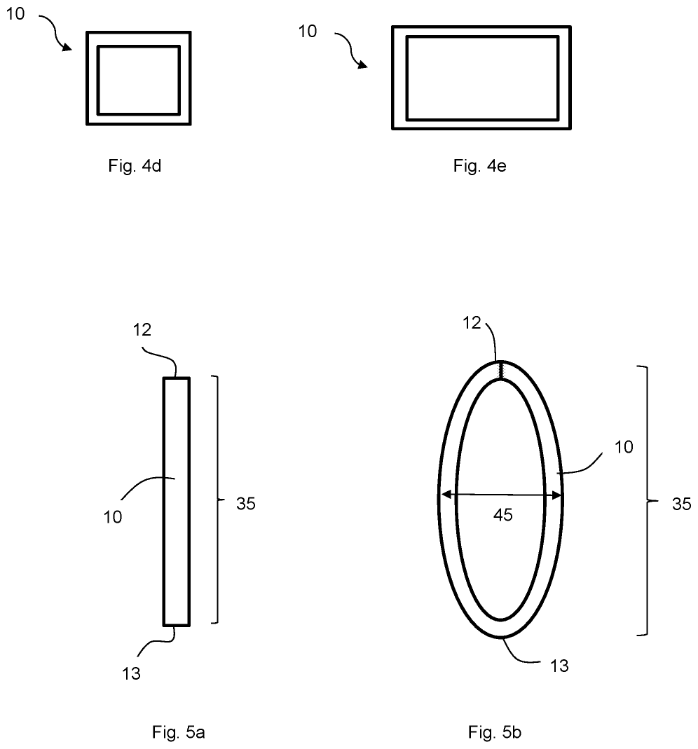

Loop central opening 11 can have any desired shape, such as (but not limited to) rounded, circular, triangular, square, rectangular, and the like, as illustrated in FIGS. 4a-4e. In some embodiments, the central opening can change as desired by the user, such as when the loop is constructed from semi-rigid materials. In other embodiments, the shape of the central opening is formed and does not change.

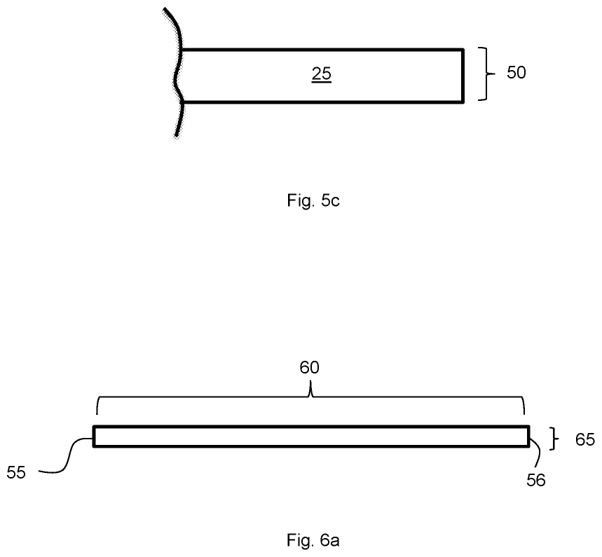

In some embodiments, loop 10 can have a length 35 of about 4-12 inches (e.g., at least/no more than about 4, 5, 6, 7, 8, 9, 10, 11, or 12 inches). The term “length” refers to the longest vertical straight-line distance from top end 12 to bottom end 13 of the loop, as shown in FIG. 5a. The loop can include width 45 of about 5-10 inches (e.g., at least/no more than about 5, 6, 7, 8, 9, or 10 inches). The term “width” refers to the longest straight-line horizontal distance of the loop (e.g., between opposing side edges), as shown in FIG. 5b. Each portion of material 25 that forms the loop can have thickness 50 of about 0.5-5 inches (e.g., at least/no more than about 0.5, 1, 1.5, 2, 2.5, 3, 3.5, 4, 4.5, or 5 inches), as shown in FIG. 5c. It should be appreciated that the length, width, and thickness of the loop can have any dimensions and can be smaller or greater than the ranges given above.

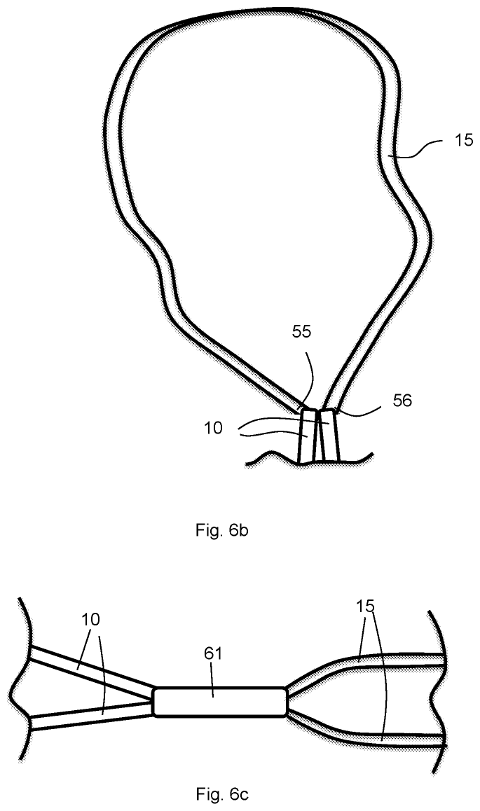

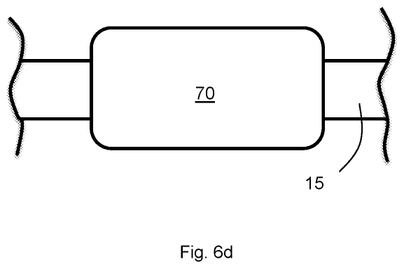

The foot loop is operatively attached to strap 15, as described above. Strap 15 of the leg sling is adapted to fit over the back and/or shoulders of a user. As shown in FIG. 6a, strap 15 can include first and second ends 55, 56 that each attach to the loop, as shown in FIG. 6b. Specifically, the loop can be attached to the strap using any conventional method, such as through the use of VELCRO®, ties, fasteners, snaps, buttons, sewing, magnets, adhesive, and the like. Thus, the loop can be permanently or releasably attached to the strap.

In some embodiments, a portion of elastic 61 can be positioned between strap 15 and loop 10, as shown in FIG. 6c. The added portion of elastic functions to increase comfort of the user. For example, the elastic provides a level of flexibility to the user when walking or moving using the disclosed device, which serves to absorb motion and shock. Elastic 61 can have any desired length, such as (but not limited to) about 0.5-6 inches. The term “elastic” refers to any material that is capable of stretching to an elongated length upon application of a biasing force without rupture or breaking, while also returning to its original length after the biasing force has been removed. It should be appreciated that elastic 61 is optional.

Strap 15 can have length 60 of about 20-60 inches or more (e.g., at least/no more than about 20, 25, 30, 35, 40, 45, 50, 55, or 60 inches). The strap also includes thickness 65 of about 1-3 inches in some embodiments. The thickness of the strap is adapted to spread the load from the leg sling over a large area of the user's shoulder(s) and/or back.

The disclosed strap can be constructed from any flexible material, allowing the user to adjust the sling as needed for comfort and to attach/remove the device. The term “flexible” refers to a material that can be easily moved, manipulated, or folded. Suitable flexible materials can include (but are not limited to) fabric, leather, polyester, elastomeric material, plastic, rubber, elastic, and the like.

In some embodiments, strap 15 can have an adjustable length, allowing proper fitting for a user. Specifically, the strap should be adjusted so that it is sufficiently short that the loop lifts the foot and leg forward, to ensure that the foot is lifted from the ground. The length of strap 15 can be altered using any adjustment 70, as shown in FIG. 6d. The adjustment can include any conventional mechanism, such as (but not limited to) clasps, fasteners, buckles, and the like. For example, one portion of strap 15 can be moved through the adjustment and secured to set the strap length. In this way, the length of strap 15 can be adjusted depending on the size of the user.

Optionally, the strap can include padding 20 to provide a level of comfort to the user's back or shoulders when the sling is in use. The padding can include any soft or cushioned material, such as fabric, cotton, foam, stuffing, filler, or combinations thereof. In some embodiments, the location of padding 20 can be adjusted along the length of the strap to provide the proper area for support. The padding can therefore be adjusted by guiding it along the length of the strap. Alternatively, the padding can be removed and positioned at a desired location along the strap.

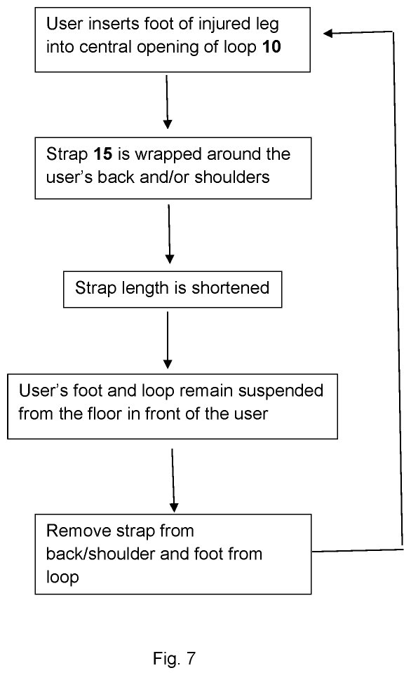

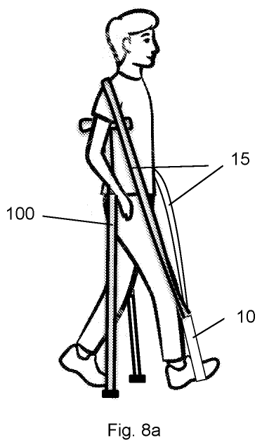

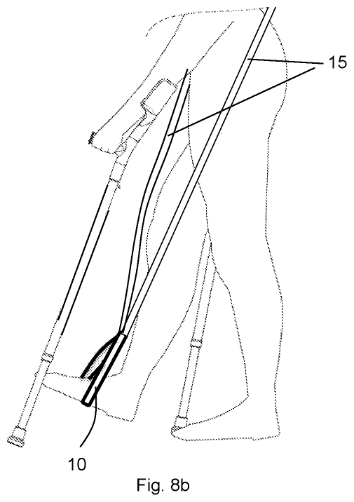

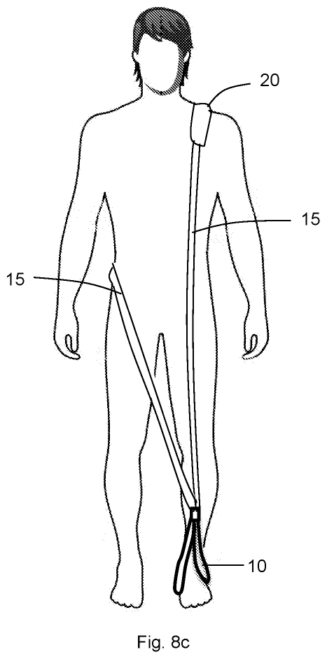

In use, the device is designed to aid orthopedic patients while they use crutches 100 or other walking implements. The user's foot is inserted into the central open portion of loop 10, as shown in the schematic of FIG. 7 and in FIGS. 8a and 8b. The bottom of the user's foot therefore comes into contact with the inner surface of loop 10. In some embodiments, the ball of the user's foot can contact loop 10. In other embodiments, the arch of the foot can be positioned within the interior of loop 10. Therefore, any lower surface of the user's foot can be used.

Either before or after the user's foot is inserted into the loop, the loop and the strap are connected. Strap 15 is then wrapped around the user's back and/or shoulders. The strap is then tightened such that the user's foot is raised from the floor. Specifically, the length of the strap is adjusted so that it is less than the length between the strap and the floor. In this way, the user's leg does not contact the floor, and is instead suspended in front of the user, as illustrated in FIGS. 8a-8c. The user's leg can be lifted about 1-10 inches from the floor, depending on user comfort levels and preference. In some embodiments, the strap is positioned over one shoulder, around the user's back and under the opposite arm or hip area to connect with the loop.

The disclosed sling can easily be used with crutches, allowing the user to move from one location to another. For example, sling 5 maintains the forward lift of the leg while using a locked extension brace or for patients with otherwise limited knee flexion. The user can then easily use the crutches, without having to manually extend their leg in front of their body.

Sling 5 can also be used following surgery where a nerve block is used. Frequently, nerve blocks are injected into the nerves of the leg prior to surgery to limit the amount of pain patients feel after surgery. However, the nerve blocks also limit a patient's ability to control the leg after surgery as they try to get home safely or use the restroom for the next 12-24 hours after the block was applied.

Because loop 10 is rigid or semi-rigid, the foot sling is also useful for lifting, moving, and limiting the unintentional movement of the leg during this time period when it feels dead and is unresponsive to muscle cues. The sling is also useful for lifting and moving the leg during the recovery phase following major surgeries for patients that require time to regain full muscle function, which is a common problem following major surgeries to the knee and leg.

When it is desired to remove sling 5, the user simply removes strap 15 from around the shoulders and/or back. The user's foot can then be easily removed from loop 10, and the device stored for later use.

Device 5 offers many advantages to the user. For example, the device is simple and easy to use, such that even children or the elderly can apply and remove the device.

Because the device raises the foot from the floor, inadvertent contact with objects on the floor are minimized or avoided, thereby reducing the chance of further damage to the foot or leg.

The disclosed device is adjustable, such that it fits users of all sizes. Thus, the device can be used with children or adults of any height and weight.

Device 5 is comfortable to wear and does not stress or strain the muscles of the leg or hip muscles to retain the leg in the forward direction.

The device is constructed from lightweight materials, thereby avoiding additional weight commonly experienced with prior art braces and supports.

The device is compact and can easily be folded or secured in a drawer or closet when not in use. Because the device can be folded, it does not take up extensive amounts of room when not being used.

Exemplary embodiments of the methods and components of the presently disclosed subject matter have been described herein. As noted elsewhere, these embodiments have been described for illustrative purposes only, and are not limiting. Other embodiments are possible and are covered by the presently disclosed subject matter. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

### EXAMPLE

### Example 1

Working in concert with an orthopedic surgeon specializing in knee and hip surgeries, sixty patients have been selected to participate in the device trial. The patients were identified because they are undergoing surgeries that will require them to use under-arm crutches for substantial periods of time during their recovery period. The patients were randomized to either a control group or the trial group. The patients in the control group received the current standard of care for their recovery period, specifically any usually prescribed crutches and braces. The trial group received that same standard of care, and additionally a foot sling device as disclosed herein. Both groups were asked to track the average number of steps that they take each week, and to fill out a once-weekly survey regarding their ease of mobility. The patients participated for six weeks or until they receive medical clearance to stop using crutches, whichever occurs first. The mobility levels of the trial group will then be compared to the mobility levels of the control group. The results are ongoing.

What is claimed is:

1. A sling for supporting the leg and foot of a user, the sling comprising:
a loop with an interior opening, sized and shaped to house the foot of a user, whereby the loop is configured to transfer the weight of the user's leg to the sling;
a strap defined by first and second ends, each end operatively attached to a top end of the loop;
wherein the strap transfers weight from the loop to the patient's shoulder, back, or both;
wherein the length of the strap is adjustable; and
wherein the strap length is less than the length between the strap and a floor, such that the loop is elevated from the floor in a forward direction relative to the user.

2. The sling of claim 1, wherein the loop is constructed from a rigid or semi-rigid material.

3. The sling of claim 1, wherein the strap includes one or more padded areas.

4. The sling of claim 1, wherein an interior surface of the loop comprises a textured region, padded lining, or both.

5. The sling of claim 1, wherein the loop central opening has a circular, oval, square, rectangular, or triangular shape.

6. The sling of claim 1, wherein the loop has a length of about 4-12 inches and the strap has a length of about 20-60 inches.

7. The sling of claim 1, wherein the strap is constructed from a flexible material.

8. A method of supporting a user's foot and leg, the method comprising:
inserting a foot of user into the loop of the sling of claim 1;
attaching each end of the strap to an end of the loop;
securing the strap around the back, shoulder, or both of the user;
adjusting the length of the strap such that the foot positioned within the loop is extended in front of the user and raised from the floor;
whereby the user's foot is supported.

9. The method of claim 8, wherein a bottom surface of the user's foot comes into contact with an inner surface of the loop.

10. The method of claim 8, wherein the foot positioned within the loop is raised about 2-10 inches.

11. The method of claim 8, wherein the user's leg remains in a locked position.

12. The method of claim 8, further comprising moving from one area to another with crutches.

13. The method of claim 8, wherein the strap includes one or more padded areas. a textured region, or both.

14. The method of claim 8, wherein the loop is permanently or releasably attached to the strap.

15. The method of claim 8, wherein the strap has a length of about 20-60 inches.

16. The method of claim 8, wherein the strap is constructed from a flexible material.

17. A kit comprising:
a plurality of loops, each loop defined by an interior opening, sized and shaped to house the foot of a user:
a plurality of straps, each strap defined by first and second ends, each end operatively attached to an end of the loop:
wherein a user selects a specific type of loop and strap for forming into a leg sling, and
wherein the strap length is less than the length between the strap and a floor, such that the loop is elevated from the floor in a forward direction relative to the user.

18. The kit of claim 17, wherein the strap includes one or more padded areas. a textured region, or both.

19. The kit of claim 17, wherein the loop has a length of about 4-12 inches and the strap has a length of about 20-60 inches.

20. A sling for supporting the leg and foot of a user, the sling comprising:
a loop with an interior opening, sized and shaped to house the foot of a user, whereby the loop is configured to transfer the weight of the user's leg to the sling;
a strap configured as a circle that passes through the loop;
wherein the strap transfers weight from the loop to the patient's shoulder, back, or both;
wherein the length of the strap is adjustable; and
wherein the strap length is less than the length between the strap and a floor, such that the loop is elevated from the floor in a forward direction relative to the user.
